(19) United States
(12) Patent Application Publication
(10) Pub. No.: US 2008/0245132 A1
(43) Pub. Date: Oct. 9, 2008

(54) METHODS OF DETECTING AND ELIMINATING TAINTED CORK WINE BOTTLE STOPPERS

(76) Inventors: Michael S. Head, Princeton, NJ (US); Mark Burrows, Newtown, PA (US)

Correspondence Address: WARD AND SMITH, P.A., 1001 COLLEGE COURT, P.O. BOX 867, NEW BERN, NC, 28563-0867, US

(21) Appl. No.: 11/697,824

(22) Filed: Apr. 9, 2007

(51) Int. Cl. G01N 33/497

(52) U.S. Cl. 73/23.34

(57) ABSTRACT

Methods are provided for detecting the presence of one or more chemical contaminants in/on a plurality of items, e.g., cork stoppers. According to one embodiment of the present invention, a method of selecting cork stoppers substantially free of cork taint is provided. The method comprises the steps of arraying and classifying one or more cork stoppers into a formatted array to enable inspecting each stopper; inspecting each cork stopper within the formatted array for the presence of cork taint using an automated means of inspection; and sorting the cork stoppers within the formatted array into either (i) a rejected group consisting of those stoppers found in the inspecting step to have cork taint or (ii) an accepted group consisting of those stoppers found in the inspecting step to be substantially free of cork taint. These methods avail of apparatus that may use detection sensor electronics that separate from nose chips such that each nose chip can be either reused or discarded after use. Testing apparatus may use multiple sensor units to simultaneously test multiple cork stoppers for chemical contaminants (e.g., TCA). These methods provide 100% testing of cork stoppers cost effectively at high speed, whether at bottling-line speed or in off-line and third-party settings.

### BACKGROUND OF THE INVENTION

1. Technical Field

The invention disclosed herein relates generally to the field of wine bottling, and more particularly to providing methods for testing cork wine bottle stoppers for the presence of an analyte such as TCA in order to prevent and/or divert tainted corks away from the wine bottling line.

2. Background Art

The wine industry produces approximately fourteen billion bottles of wine per year. The bottled wines range in price from inexpensive table wines to very expensive, high-quality wines. The more expensive wines (i.e., from fifty dollars to thousands of dollars per bottle) are typically produced by a small number (presently, about two thousand) high-end wineries that produce 200,000 to 80 million bottles of wine each per year.

Most bottled wines, about 80 percent, both inexpensive and expensive, are sealed with cork stoppers. Cork stoppers include natural cork stoppers punched from strips of bark and less expensive molded or extruded agglomerated cork with natural cork discs on each end. Wine makers generally prefer cork stoppers for sealing their bottles to maintain the traditional wine-opening experience that consumers expect. Unfortunately, the use of cork stoppers can adversely affect the taste of wine, a characteristic commonly referred to as “cork taint.” Cork taint describes the “off” smell and taste imparted to wine from chemical contaminants, most commonly 2,4,6-trichloroanisole (TCA) in the cork stopper. While TCA is the most common chloroanisole contributing to cork taint, other chloroanisoles including tetrachloroanisole (TeCA) and pentachloroanisole (PCA) can contribute to cork taint.

The incidence of cork taint is sporadic and random, estimated to affect 1-2% of bottled wines. Since cork taint takes effect after bottling, it cannot be detected until after a bottle has been opened. Cork taint manifests as very undesirable aroma and flavor characters that are imparted to bottled wines following contact with the cork. There is nothing more offensive and embarrassing for wine consumers and producers alike than for their wine to be rated as “spoiled.” For consumers, opening a cork-tainted bottle of wine can be socially embarrassing, particularly if it is an expensive bottle of wine. For wine collectors, the 1-2% incidence of cork taint, however, imparts uncertainty about their entire wine collection. For wine producers, cork-tainted wine damages their reputation, causing consumers to question the integrity, quality, and price of their wine. Thus, there exists a need for a means to ensure that cork stoppers used to bottle wines are free of TCA taint.

As discussed hereinabove, the chemical compound contributing most significantly to cork taint is TCA, which is implicated in more than 80% of cork-tainted wines. TCA is present in nature. Presence of TCA in cork stoppers is the result of both nature and complex chemical mechanisms, including the conversion of chlorophenols to chloroanisole by common microorganisms, such as fungi, in the presence of moisture. Cork taint results from interaction between naturally occurring fungi in the tree bark and chlorine, a chemical commonly used to sanitize cork in production. Cork, like any other wine input, therefore demands exhaustive quality control.

A leading cork producer, Amorim of Portugal, (www.corkfacts.com) has taken a concerted approach, called ROSA, which it describes as “a proprietary cleaning process developed by Amorim as a result of intensive R&D effort over more than three years involving thousands of experiments and chemical analysis and a major process re-engineering program,” to eliminate the natural incidence of TCA in cork stoppers. The website goes on the report that the ROSA program “achieved TCA reductions in cork granules ranging from 75 to 92 percent.” Amorim also highlights that it spends $US 6 million per year in R&D, to eliminate TCA from cork. Yet despite this sizable investment and this significant level of TCA reduction, the full assurance that corks used in wine bottling are free of TCA cannot be made. Amorim also advocates vertical integration of the cork production process, from tree to manufacturing, i.e. purchasing the cork-tree forests for maximum control over factors commonly leading to TCA incidence, such as use of certain pesticides. Yet TCA occurs in nature thus cannot be eliminated by even to most effective of forestry and manufacturing processes. Thus there exists a need to detect TCA in cork stoppers even after their manufacture, no matter how precise the manufacturing and inspection process.

Indeed, quality assurance at every step of the cork stopper manufacturing process is a major focus of the cork industry. This concern has led to the implementation of the “International Code of Cork Stoppers Manufacturing Practices.” The code establishes quality-control standards throughout the production process and aims to provide guarantees to cork suppliers, wine producers, and bottlers that they have a product that is free from contamination. Yet all cork stoppers are not free of contamination. Thus, there exists a need for a testing process that provides 100% testing of cork stoppers for TCA content prior to bottling.

In addition, premium cork suppliers also insist on rigorous quality-control testing of their cork stoppers for TCA. Current industry practices for quality-control testing of cork stoppers include sensory-based methods (i.e., olfactory detection or human experts) and chemical analysis (e.g., cork soaks and gas chromatography/mass spectroscopy). However, these testing procedures are limited to testing sample batches of cork stoppers (e.g., statistical sampling). For example, for every 100 million or more cork stoppers produced, only a half-million to one million are tested for TCA. The batch sampling approach does not eliminate the possibility that a TCA-tainted cork will be undetected during quality-control testing and subsequently used by a wine producer or bottler. Thus, there exists a need for a testing process that provides 100% testing of cork stoppers for TCA content prior to bottling.

Another limitation of current testing methods is that they are expensive and time consuming. Further, sensory-based methods that rely on human experts are subjective, variable and exhaustible. Thus, there exists a need for a low-cost, automated testing process that provides 100% testing of cork stoppers for TCA prior to bottling.

The wine industry, seeking to increase consistency and consumer loyalty, has investigated alternative quality-control procedures. One alternative is the application of electronic nose technology to quality-control testing at all stages of wine production, e.g., bottling. An electronic nose is a sensing device capable of producing a fingerprint of specific odors. Current technology includes electronic noses that use odor-reactive polymer sensor arrays and a pattern-recognition system (i.e., e-Nose) and gas chromatography coupled to surface acoustic wave sensors (i.e., z-Nose). In one example of polymer sensor arrays, the electronic nose uses a one-inch-square micro-electrical mechanical systems (MEMS) chip containing 32 pinhead-sized receptors forming a sensor array. The receptors are constructed from a conductive carbon black material blended with specific nonconductive polymers (manufactured by Cyrano Sciences, Inc., Pasadena, Calif.). When the MEMS chip is exposed to a specific vapor, a corresponding receptor expands, temporarily breaking some of the connections between the carbon black pathways and thereby increasing the electrical resistance in the sensor. Signals from the sensors are electronically processed by a microprocessor that interprets the data by using the pattern-recognition system to identify and/or quantify a specific odor contained in the vapor.

Application of electronic nose technology to quality-control monitoring of agricultural products is exemplified in U.S. Pat. No. 6,450,008 to Sunshine et al., entitled “Food applications of artificial olfactometry.” The Sunshine et al. patent describes a method and device for evaluating agriculture products and, more particularly, for assessing and monitoring the quality of food products by using electronic noses. The quality control monitoring device includes two sensor arrays for comparative monitoring of an agricultural product, e.g., before and after a processing step such as blending or mixing, or detection of a contaminant (e.g., a microorganism) relative to a clean sample. However, the quality-control monitoring device is a single device that typically requires three minutes to obtain a result and to cycle to the next measurement, thus limiting the number of measurements that can be determined by a single device. Further, the existing devices are expensive, which precludes purchasing multiple instruments to achieve 100% testing of a product in a production process at bottling-line speed. Thus, there exists a need for a means to test 100% of all corks cost-efficiently at bottling-line speed.

The introduction of a new technology platform (e.g., electronic nose technology) into an existing industry (e.g., the wine industry) is often a difficult and expensive process. Often, a new technology platform is implemented by high-end or specialty producers (e.g., high-end wine producers), for which the costs associated with the production of a quality product are generally higher and the benefits provided by the new technology are initially greater. However, this approach neglects the general consumer market (e.g., inexpensive table wines), in which the volume of products consumed offers greater potential returns. Thus, there exists a need for a means to test 100% of all corks at bottling-line speed that is cost-efficient and scalable to the general consumer market.

### BRIEF SUMMARY OF THE INVENTION

According to one embodiment of the present invention, a method of selecting cork stoppers substantially free of cork taint is provided. The method comprises the steps of arraying and classifying one or more cork stoppers into a formatted array to enable inspecting each stopper; inspecting each cork stopper within the formatted array for the presence of cork taint using an automated means of inspection; and sorting the cork stoppers within the formatted array into either (i) a rejected group consisting of those stoppers found in the inspecting step to have cork taint or (ii) an accepted group consisting of those stoppers found in the inspecting step to be substantially free of cork taint.

It is therefore an object to provide methods for identifying and eliminating tainted corks prior to and/or during beverage bottling operations, such as wine bottling, at bottling-line speeds, without slowing the bottling process.

An object of the present invention having been stated hereinabove, and which is addressed in whole or in part by the present invention, other objects will become evident as the description proceeds when taken in connection with the accompanying drawings as best described hereinbelow.

### BRIEF DESCRIPTION OF THE DRAWINGS

FIG. 1 is a flow diagram of a method of inspecting corks at a cork production site;

FIG. 2 is a flow diagram of a method of inspecting cork in line with winery bottling;

FIG. 3 is a flow diagram of a method of inspecting cork off line at a winery; and

FIG. 4 is a flow diagram of a method of inspecting cork at a third-party site.

### DETAILED DESCRIPTION OF THE INVENTION

While the present description focuses on the use of cork stopper as a preferred seal and closure for bottles of wine, the skilled artisan will appreciate that the methods taught herein are equally applicable to a selection of cork stoppers for sealing bottles of other beverages such as whiskey and liqueurs as well as food products such as balsamic vinegar. As used herein, the term “third-party site” means a location of a bottling operation under the primary control of a party other than the party responsible for inspection and selection of cork stoppers. For example, the methods taught herein might be carried out by a party under contract to a beverage bottler at the bottler's place of business. As used herein, the terms device, apparatus, and machine are synonymous.

As used herein, the term “cork taint” means halogenated anisoles including trichloroanisole (TCA), tribromoanisole (TBA), tetrachloroanisole (TeCA), and pentachloroanisole (PCA), as well as the family of halogentated molds and analytes associated with the production of halogenated anisoles in cork. TCA, TBA, TeCA, and PCA are all chemically related haloanisole compounds that differ in their number of halogen atoms. Each compound has a similar odor but possesses different sensory thresholds.

The origin of haloanisoles is often attributed to the biodegradation of halophenols by molds. Trichlorophenol (TCP), tetrachlorophenol (TeCP), pentachlorophenol (PCP) and tribromophenol (TBP) are converted to TCA, TeCA, PCA and TBA respectively. TCP in wineries is often traced to the former use of bleach as a sanitizer. TeCP, PCP and TBP are wood preservatives. Each of these compounds, or combinations thereof, can be found in winery wood materials. TBP, also used in flame retardants, can be present in plastics and other polymers and may also result from the use of bromine as a sanitizer. The origin of TCA in natural corks is still debated.

Generally, “cork taint” results from the migration of TCA from cork in bottled wine, giving off-odors described as “musty”, “wet cardboard” or “moldy”. Other haloanisoles however, can have a similar effect on wine. For both bulk and bottled wine, the relative concentrations of TCA, TeCA, PCA and TBA often suggest a possible contamination source. For example, the presence of pentachlorophenol-treated wood in the cellar would be suspected when TeCA and PCA are the predominant haloanisoles. With bottled wines, bottle-to-bottle variability provides additional information. Significant bottle variability and the predominance of TCA suggest that the corks may be the contamination source. Analyzing wine at bottling is highly recommended. It is the only way to confirm if contamination occurred before or after bottling. If a sample taken at bottling is positive, causes of contamination in the cellar can be investigated.

While the primary test method taught herein targets TCA, the major type of cork taint, skilled artisans will recognize that other halogenated molds and analytes may indicate the presence of TCA and vise versa. This aspect enables detection of TCA indirectly by detecting these other halogenated molds and analytes.

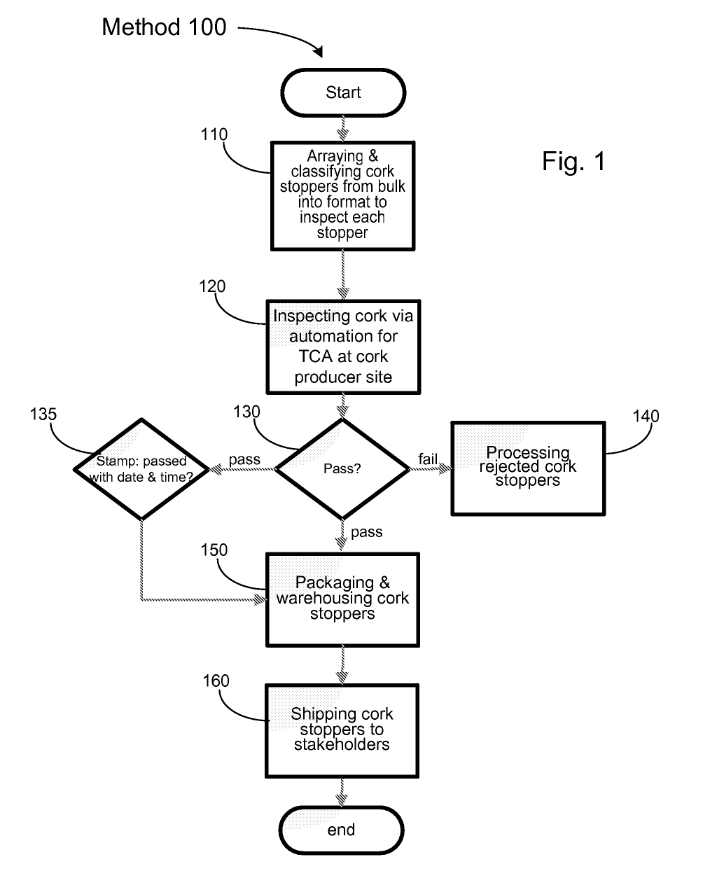

Referring now to FIG. 1, method 100 is illustrated and describes an automated 100 percent cork-inspection process at the cork manufacturer's facility. Method 100 includes the following steps.

Arraying step 110 includes arraying and classifying cork stoppers from bulk into a format to inspect each stopper. That is, the stoppers must be arranged in an array or pattern acceptable to automated inspection devices, and thus, enabling the automated device to inspect the stoppers. In arraying step 110, corks are arrayed from bulk storage for individual automated inspection, such as with the system CorkInspect of Barcelona, Spain which “. . . allows defects detection and quality classification of cork stoppers in a minimum processing error and without any manual inspection” (www.cvc.uab.es). The CorkInspect process arrays each cork severally in a web for visual inspection via automated visual system such as machine vision, then classifies each cork, such as Grade A, B, C, per Cork Quality Council standards. Present systems look only visually for presence/absence of various defects, resulting in classification of each cork as Grade A, B, or C, according to criteria established by the wine industry's Cork Quality Council (www.corkqc.com) and adopted by its members. The classification is typically as follows:
Visual Grade A—These are corks with top quality visual appearance—excellent surfaces, with no major visual flaws and few small ones.
   No holes or pores which exceed 2 mm.
   No cracks originating at the ends which exceed 11% of cork length.
   No cracks in the body of the cork to exceed 18% of cork length.
   All cracks must be tight and not open.
   No horizontal cracks.
   No worm holes, hardwood, belly spots, or greenwood.
   Several narrow and shallow lenticels are acceptable if they are free of dust and particles.
Visual Grade B—These are corks of good visual appearance with no major visual flaws and with surface visual flaws of no depth or substance.
   No holes or pores which exceed 5 mm.
   No cracks originating at the ends which exceed 18% of cork length.
   No cracks in the body of the cork to exceed 25% of cork length.
   All cracks must be tight and not open.
   Lenticels and horizontal cracks must not open up when ends of the corks are bent.
   No greenwood.
   No angled or deformed corks.
   Very small chips and lateral worm activity in the middle of the body of the cork may be acceptable.
   Lenticels at ends must not be wide or deep and should be free of dust and particles.
Visual Grade C—These are corks of average visual appearance with one or more major visual flaws which will be of cosmetic nature only. Thus they may be aesthetically unappealing, but functional.
   No cracks, channels, hardwood or belly spots which exceed 55% of cork length.
   Lenticels and horizontal cracks on body may open up when ends of the corks are bent.
   Greenwood to 55% of cork length is acceptable unless severe depth or width.
   Large chips are acceptable.
   No worm activity from end to side which exceed 55% of cork length.
   No dry years which exceed 55% of cork length.
   There may be heavy, but not continuous porosity.

Referring further to FIG. 1, method 100 proceeds to inspecting step 120 which includes inspecting cork via automation for TCA at the cork producer site. In inspecting step 120, the capability of inspecting each cork at the cork producer site may be introduced, such as by adding it to the inspection apparatus of arraying step 110 or use of a discrete apparatus. The cork producer also may use this apparatus to inspect corks produced by others for revenue such as inspection fees, business intelligence on competitive cork volumes produced, and the like. A representative method and apparatus of detecting TCA are explained by Head et al. in U.S. Pat. No. 7,010,956, including its FIG. 1, which is incorporated herein by reference.

Method 100 then proceeds to pass inspection inquiry step 130. In inquiry step 130, corks which fail inspection for presence of TCA in inspecting step 120 proceed to rejection step 140 where they may be quickly diverted well away from other corks to minimize potential for contamination of other corks. Rejected corks may be used to manufacture other products such as cork board. Corks which pass inspection for presence of TCA in inspecting step 120 proceed to approval step 135. In approval step 135, corks passing inspection in inspecting step 120 may be stamped to denote absence of TCA, date and time of inspection, inspecting party, and/or other associated information enabling traceability through the supply chain. Such traceability enables identification and elimination of TCA sources. Corks from inquiry step 130 and/or approval step 135 proceed to packaging step 150 including the packaging and warehousing of cork stoppers.

In packaging step 150, corks from inquiry step 130 and/or approval step 135 are packaged and stored in a manner to ensure quality standards throughout the supply chain. Such quality standards are consistent with the Cork Quality Council's “Recommended Corking Practices for Wineries” which is summarized as follows:
I. CORKER JAW TYPE
   1. The 4 segment sliding roller or iris jaw type cork compression system is recommended. Roller or iris type jaws tend to cause wrinkles in the cork which can cause leaking.
II. CORKER MAINTENANCE
   1. Corking machines are maintained to manufacturers recommended standards at all times.
   2. Smooth action in compression stage.
   3. No nicks or other damage to the jaw segments.
   4. Good alignment and seal of bottle neck in centering bell.
   5. Properly centered plunger.
   6. Daily cleaning and sanitation of cork handling surfaces; i.e. hopper, feed tube, orienter, and jaws.
   7. A 24 mm cork should not be compressed to less than 16 mm.
III. CORK HANDLING AND STORAGE
   1. Do not open plastic cork bags until immediately before loading corks into the loading corking machine. No bags containing corks should be left open for any reason.
   2. Corks recovered from the corking machine after the bottling is completed should be returned to the plastic bag or another closable container “dosed” with sulfur dioxide gas (vapor) and sealed tightly. CQC companies can provide this service.
   3. Corks should be stored in a cool dry location, not in a bottling room, barrel storage area, or chemical storage area. The temperature should be 55 to 70 degrees Fahrenheit and the humidity 50 to 70 percent.
IV. MOISTURE CONTENT
   1. New shipments of cork, as well as corks that have been stored for extended periods of time, should be checked for moisture content before use. Corks below 5% average moisture level should be discarded or returned to the supplier for rehydration and sterile packaging.
   2. Corks with an average moisture content of over 8% should be regarded with suspicion as such moisture level could support mold growth.
V. INTERNAL BOTTLE PRESSURE
   1. Wine temperature should be between 60-70 degrees Fahrenheit. If lower temperatures are used then the fill point should be adjusted down to compensate for expansion in the bottle when room temperature is reached. Be sure to maintain legal fill volume.
   2. If the fill pint is too high, less vacuum can be achieved.
   3. The vacuum system should be well controlled and maintained. Gauges which continuously display vacuum status at the corking head and request (each ½ hour) on line QC of corked bottles (pierce test) are highly recommended.
   4. Bottles should remain upright for 24 hours after corking. It is recommended that the above elements be combined to produce a net effect of no more than 3 psi internal bottle pressure at 68 degrees.

Method 100 then proceeds to shipping step 160 which includes shipping cork stoppers to stakeholders. In shipping step 160, packaged and stored corks from packaging step 150 may be delivered, shipped, and or otherwise conveyed to supply chain stakeholders such as customers, wineries, distributors, bottlers, and the like. Method 100 then ends.

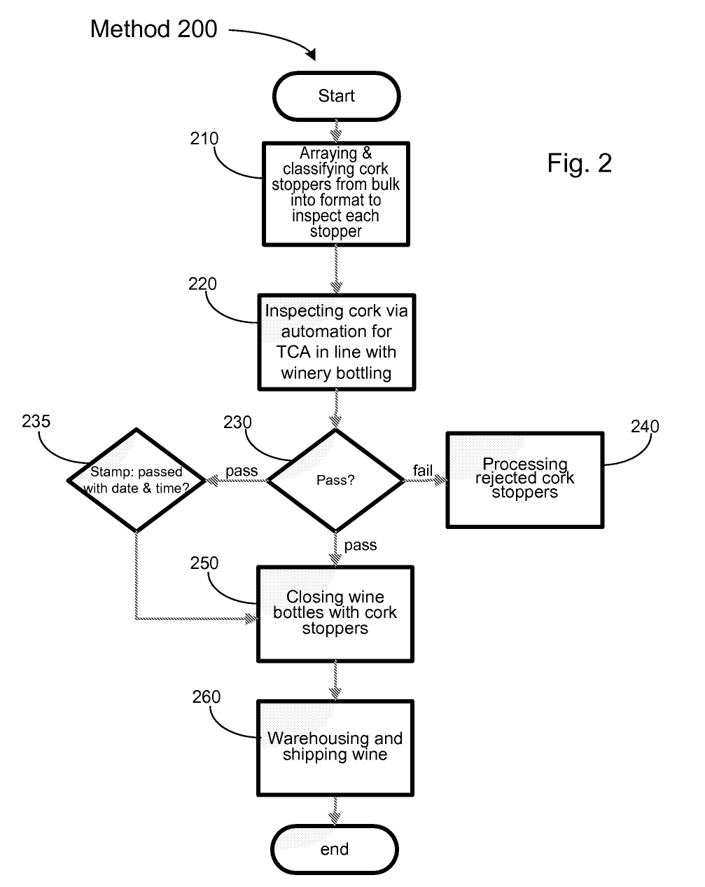

Wine producers and/or bottlers may prefer to place 100 percent cork inspection apparatus on their bottling lines such as to meet their specific quality control standards, to implement proprietary inspection and/or bottling processes which may yield competitive advantage, and the like. With reference to FIG. 2, method 200 is illustrated and describes an automated 100 percent cork inspection process in line with winery bottling. Method 100 includes the following steps.

Arraying step 210 includes arraying and classifying cork stoppers from bulk into a format to inspect each stopper. In arraying step 210, corks are arrayed from bulk storage for individual automated inspection, typically with automated systems and according to standards as discussed hereinabove in relation to arraying step 110.

Method 200 then proceeds to inspecting step 220 for inspecting cork via automation for TCA in line with wine bottling. In inspecting step 220, the capability of inspecting each cork in line with wine bottling may be introduced, such as by adding it to the inspection apparatus of arraying step 210 or use of a discrete automated apparatus. The wine bottler also may use this apparatus to inspect corks produced for other bottlers for revenue such as inspection fees, business intelligence on competitive wine volumes produced, and the like. A representative method and apparatus of detecting TCA are explained by Head et al. in U.S. Pat. No. 7,010,956, including its FIG. 1, which is incorporated herein by reference.

Method 200 then proceeds to pass inspection inquiry step 230. In inquiry step 230, corks which fail inspection for presence of TCA in inspecting step 220 proceed to rejection step 240 where they may be quickly diverted well away from other corks to minimize potential for contamination of other corks. Rejected corks may be used to manufacture other products such as cork board. Corks which pass inspection for presence of TCA in inspecting step 220 proceed to approval step 235. In approval step 235, corks passing TCA inspection in inspecting step 220 may be stamped to denote absence of TCA, date and time of inspection, inspecting party, and/or other associated information enabling traceability through the supply chain. Such traceability enables identification and elimination of TCA sources. Corks from inquiry step 230 and/or approval step 235 proceed to closing step 250 including closing the wine bottles with cork stoppers.

In closing step 250, corks from inquiry 230 and/or approval step 235 proceed to the station in the bottling line for insertion into wine bottles and are inserted into wine bottles at customary wine-bottling speed, completing the bottle-closure process.

Method 200 then proceeds to shipping step 260 which includes warehousing and shipping of the wine bottles. In shipping step 260, bottled wine from closing step 250 may be stored in customary wine-maker conditions, then at the appropriate time may be delivered, shipped, and/or otherwise conveyed to supply chain stakeholders such as customers, distributors, retailers, and the like. Method 200 then ends.

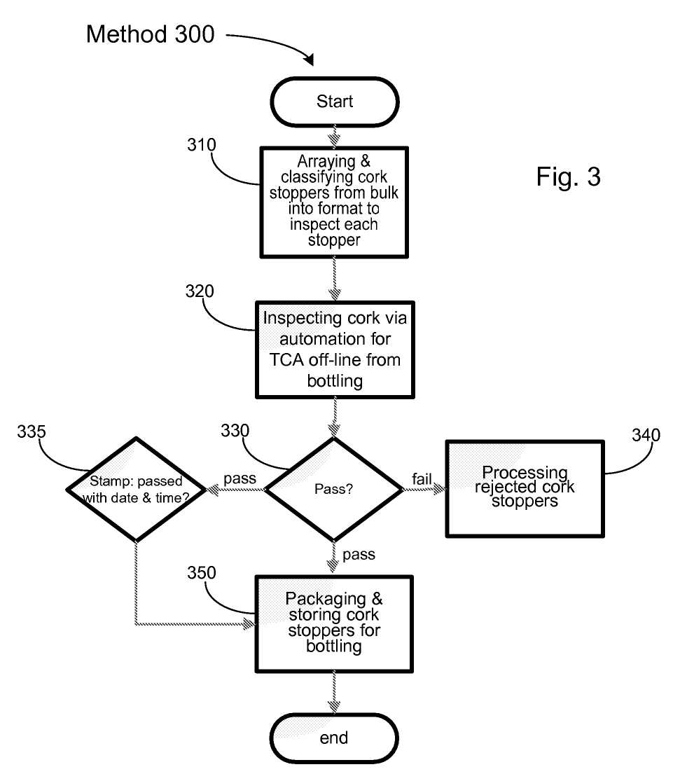

Wine producers and/or bottlers may prefer to place 100 percent cork inspection apparatus off-line from their bottling operations such as to meet their specific quality control standards, to implement proprietary inspection and/or bottling processes which may yield competitive advantage, and the like. With reference to FIG. 3, method 300 is illustrated and describes an automated 100 percent cork inspection process off-line from winery bottling. Method 300 includes the following steps.

Arraying step 310 includes arraying and classifying cork stoppers from bulk into a format to inspect each stopper. In arraying step 310, corks are arrayed from bulk storage for individual automated inspection, typically with automated systems and according to standards as discussed hereinabove in relation to arraying step 110.

Method 300 then proceeds to inspecting step 320 for inspecting cork off-line from bottling. In inspecting step 320, the capability of automatically inspecting each cork off-line from bottling may be introduced, such as by adding it to the inspection apparatus of arraying step 310 or use of a discrete automated apparatus. The wine bottler also may use this apparatus to inspect corks for wine produced by others for revenue such as inspection fees, business intelligence on competitive cork volumes produced, and the like. A representative method and apparatus of detecting TCA are explained by Head et al. in U.S. Pat. No. 7,010,956, including FIG. 1, which is incorporated herein by reference.

Method 300 then proceeds to pass inspection inquiry 330. In inquiry step 330, corks which fail inspection for presence of TCA in inspecting step 320 proceed to rejection step 340 where they may be quickly diverted well away from other corks to minimize potential for contamination of other corks. Rejected corks may be used to manufacture other products such as cork board. Corks which pass inspection for presence of TCA in inspecting step 320 proceed to approval step 335. In approval step 335, corks passing TCA inspection in inspecting step 320 may be stamped to denote absence of TCA, date and time of inspection, inspecting party, and/or other associated information enabling traceability through the supply chain. Such traceability enables identification and elimination of TCA sources. Corks from inquiry step 330 and/or approval step 335 proceed to packaging step 350 including packaging and storing cork stoppers for bottling.

In packaging step 350, corks from inquiry step 330 and/or approval step 335 are packaged and stored in a manner to ensure quality standards until needed in the bottling operation, such as consistent with the Cork Quality Council's Recommended Corking Practices for Wineries, explained hereinabove with reference to packaging step 150. An example of such a manner of packaging is hermetic sealing in plastic bags/wraps. An example of such a manner is storage of cork packages in a cool, dry location, not in a bottling room, barrel storage area, and/or chemical storage area. Temperature for storage is typically in the range of 55 to 70 degrees Fahrenheit and the humidity for storage is typically in the range of 50 to 70 percent. Method 300 then ends.

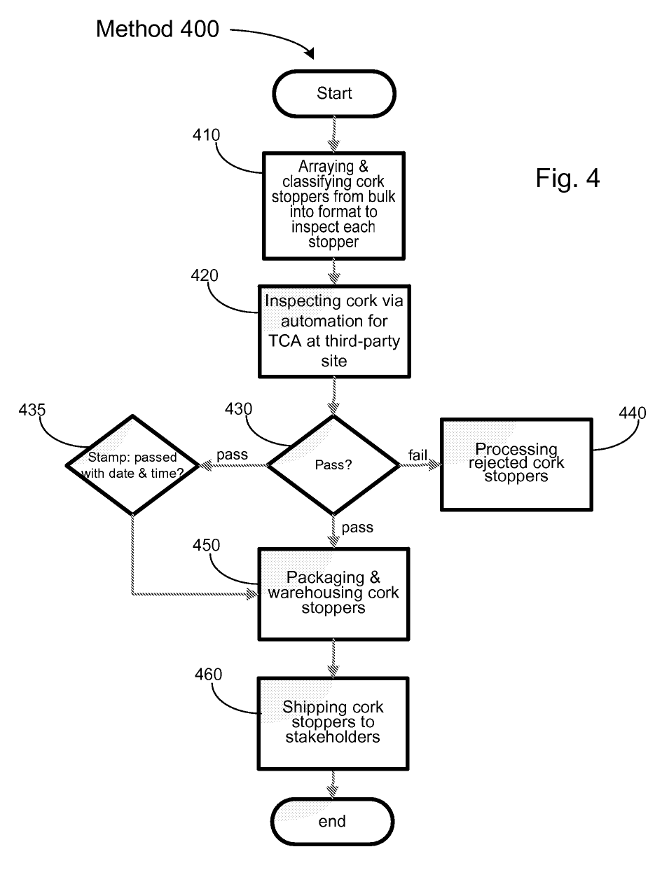

Factors such as economies of scale may call for a third-party 100-percent automated cork inspection with a third party who may be the cork producer and/or other supplier specializing in cork inspection. Wine producers and/or bottlers may prefer placement of 100-percent automated cork inspection apparatus with a vendor for such reasons as to avoid capital investment in such automated inspection apparatus, to meet their specific quality control standards, to implement proprietary inspection and/or bottling processes which may yield competitive advantage, and the like. With reference to FIG. 4, method 400 is illustrated and describes an automated 100 percent cork inspection process which resides with a third-party supplier. Method 400 includes the following steps.

Arraying step 410 includes arraying and classifying cork stoppers from bulk into a format to inspect each stopper. In arraying step 410, corks are arrayed from bulk storage for individual automated inspection, typically with automated systems and according to standards as discussed hereinabove in relation to arraying step 110.

Method 400 then proceeds to inspecting step 420 for inspecting cork via automation for TCA at a third-party site. In inspecting step 420, the capability of inspecting each cork at the cork producer site may be introduced, such as by adding it to the inspection apparatus of arraying 410 or use of a discrete apparatus. The cork producer also may use this apparatus to inspect corks produced by others for revenue such as inspection fees, business intelligence on competitive cork volumes produced, and the like. A representative method and apparatus of detecting TCA are explained by Head et al. in U.S. Pat. No. 7,010,956, including its FIG. 1, which is incorporated herein by reference.

Method 400 then proceeds to pass inspection inquiry step 430. In inquiry step 430, corks which fail inspection for presence of TCA in inspecting step 420 proceed to rejection step 440 where they may be quickly diverted well away from other corks to minimize potential for contamination of other corks. Rejected corks may be used to manufacture other products such as cork board. Corks which pass inspection for presence of TCA in inspecting step 420 proceed to approval step 435. In approval step 435, corks passing TCA inspection in inspecting step 420 may be stamped to denote absence of TCA, date and time of inspection, inspecting party, and/or other associated information enabling traceability through the supply chain. Such traceability enables identification and elimination of TCA sources. Corks from inquiry step 430 and/or approval step 435 proceed to packaging step 450 including the packaging and warehousing of cork stoppers.

In packaging step 450, corks from inquiry step 430 and/or approval step 435 are packaged and stored in a manner to ensure quality standards throughout the supply chain, such as consistent with the Cork Quality Council's Recommended Corking Practices for Wineries, explained hereinabove with reference to packaging step 150. An example of such a manner of packaging is hermetic sealing in plastic bags/wraps. An example of such a manner is storage of cork packages in a cool, dry location, not in a bottling room, barrel storage area, and/or chemical storage area. Temperature for storage is typically in the range of 55 to 70 degrees Fahrenheit and the humidity for storage is typically in the range of 50 to 70 percent.

Method 400 then proceeds to shipping step 460 which includes shipping cork stoppers to stakeholders. In shipping step 460, packaged and stored corks from packaging step 450 may be delivered, shipped, and or otherwise conveyed to supply chain stakeholders such as customers, wineries, distributors, bottlers, and the like. Method 400 then ends.

It will be understood that various details of the present invention may be changed without departing from the scope of the present invention. Furthermore, the foregoing description is for the purpose of illustration only, and not for the purpose of limitation, as the present invention is defined by the claims as set forth hereinafter.

What is claimed is:

1. A method of selecting cork stoppers substantially free of cork taint comprising the following steps:
(a) arraying and classifying one or more cork stoppers into a formatted array to enable inspecting each stopper;
(b) inspecting each cork stopper within the formatted array for the presence of cork taint using an automated means of inspection; and
(c) sorting the cork stoppers within the formatted array into either:
(i) a rejected group consisting of those stoppers found in step (b) to have cork taint; or
(ii) an accepted group consisting of those stoppers found in step (b) to be substantially free of cork taint.

2. The method of claim 1 wherein cork taint is a halogenated anisole.

3. The method of claim 2 wherein the halogenated anisole is TCA.

4. The method of claim 1 wherein the automated means comprises a computer controlled inspecting device that uses electronic sensors to detect the presence of cork taint and a method for its employment.

5. The method of claim 4 wherein the computer controlled inspecting device comprises the apparatus and method taught by Head et al. in U.S. Pat. No. 7,010,956.

6. The method of claim 1 wherein the cork stoppers of the rejected group are quickly segregated from the cork stoppers of the accepted group.

7. The method of claim 1 wherein cork stoppers of the accepted group are stamped to denote:
(a) that they are substantially free of cork taint;
(b) the date and time they were inspected; and
(c) information to enable them to be tracked in storage, shipment, and future applications.

8. The method of claim 1 wherein the cork stoppers are to be used to close beverage bottles.

9. The method of claim 8 wherein the beverage bottles are wine bottles.

10. The method of claim 1 wherein the cork stoppers have previously been inspected or are post inspected for other than cork taint.

11. A method of selecting cork stoppers substantially free of cork taint in line with a beverage bottling operation comprising the following steps:
(a) arraying and classifying one or more cork stoppers into a formatted array to enable inspecting each stopper;
(b) inspecting each cork stopper within the formatted array for the presence of cork taint using an automated means of inspection within a beverage bottling line;
(c) sorting the cork stoppers within the formatted array into either:
(i) a rejected group consisting of those stoppers found in step (b) to have cork taint; or
(ii) an accepted group consisting of those stoppers found in step (b) to be substantially free of cork taint; and
(d) closing bottles previously filled with beverage by inserting cork stoppers from the accepted group into the bottles.

12. The method of claim 11 wherein the automated means comprises a computer controlled inspecting device that uses electronic sensors to detect the presence of cork taint and a method for its employment.

13. The method of claim 12 wherein the automated means comprise the apparatus and method taught by Head et al. in U.S. Pat. No. 7,010,956.

14. The method of claim 11 wherein the cork stoppers of the rejected group are quickly segregated from the cork stoppers of the accepted group.

15. The method of claim 11 wherein, in an additional step between steps (c) and (d), cork stoppers of the accepted group are stamped to denote:
(a) that they are substantially free of cork taint;
(b) the date and time they were inspected; and
(c) information to enable them to be tracked.

16. The method of claim 11 wherein the cork stoppers are to be used to close beverage bottles.

17. The method of claim 16 wherein the beverage bottles are wine bottles.

18. The method of claim 11 wherein after step (d) the filled and closed bottles are stored and then shipped.

19. A method of selecting cork stoppers substantially free of cork taint for beverage bottles offline of a beverage bottling operation comprising the following steps:
(a) arraying and classifying one or more cork stoppers into a formatted array to enable inspecting each stopper;
(b) inspecting each cork stopper within the formatted array for the presence of cork taint using an automated means of inspection offline of a beverage bottling line;
(c) sorting the cork stoppers within the formatted array into either:
(i) a rejected group consisting of those stoppers found in step (b) to have cork taint; or
(ii) an accepted group consisting of those stoppers found in step (b) to be substantially free of cork taint; and
(d) closing bottles previously filled with beverage by inserting cork stoppers from the accepted group into the bottles.

20. The method of claim 19 wherein, in an additional step between steps (c) and (d), cork stoppers of the accepted group are stamped to denote:
(a) that they are substantially free of cork taint;
(b) the date and time they were inspected; and
(c) information to enable them to be tracked.

21. The method of claim 19 wherein the automated means comprises a computer controlled inspecting device that uses electronic sensors to detect the presence of cork taint and a method for its employment.

22. The method of claim 21 wherein the automated means comprise the apparatus and method taught by Head et al. in U.S. Pat. No. 7,010,956.

23. The method of claim 19 wherein the cork stoppers of the rejected group are quickly segregated from the cork stoppers of the accepted group.

24. The method of claim 19 wherein the cork stoppers are to be used to close beverage bottles.

25. The method of claim 24 wherein the beverage bottles are wine bottles.

26. The method of claim 19 wherein after step (d) the filled and closed bottles are stored and then shipped.

27. The method of claim 19 wherein one or more of step (a) through (d) is preformed at a third party site.
